(19) United States
(12) Patent Application Publication
(10) Pub. No.: US 2015/0258140 A1
(43) Pub. Date: Sep. 17, 2015

(54) METHODS FOR TREATING CANCER WITH REDUCED RENAL TOXICITY

(71) Applicants: Teni Boulikas, Alimos (GR); George Stathopoulos, Athens (GR)

(72) Inventors: Teni Boulikas, Alimos (GR); George Stathopoulos, Athens (GR)

(73) Assignee: Teni Boulikas, Alimos, Attiki (GR)

(21) Appl. No.: 14/421,392

(22) PCT Filed: Aug. 13, 2012
(86) PCT No.: PCT/US2012/050630

§ 371 Date: May 12, 2015

(51) Int. Cl. A61K 33/24; A61K 9/107; A61K 47/24; A61K 45/06

(52) U.S. Cl. CPC A61K 33/24; A61K 45/06; A61K 9/107; A61K 47/24

(57) ABSTRACT

A method is provided for inhibiting the growth of a tumor in a cancer patient or treating a cancer patient, wherein the cancer patient has renal insufficiency. The method requires the administration of an effective amount of Lipoplatin. A second chemotherapeutic drug can also be administered to the patient. The second chemotherapy can be administered prior to or after the Lipoplatin therapy or simultaneously.

### BACKGROUND

The present invention relates generally to the field of solid tumors that are responsive to platinum therapy.

Cisplatin has been in use for over 30 years and has been demonstrated to be an effective agent against a number of malignancies, including lung, ovarian, head and neck, gynecological, testicular and urothelial cancers. Although cisplatin is one of the most significant and effective anticancer agents, its toxicity is often an inhibiting factor preventing the continuation of treatment courses. The main side effect is renal toxicity (renal failure). Other adverse reactions have included nausea and vomiting, asthenia and neurotoxicity.

Over the last 15-20 years, there has been an extensive effort to produce other agents as a substitute for cisplatin. The main substitutive agent was the CDDP analogue, carboplatin. Moreover, in certain malignancies other new agents, including taxanes (paclitaxel, docetaxel) and gemcitabine and vinorelbine, have been tested. Renal toxicity was avoided with the use of these agents, but other side effects, including myelotoxicity, were observed. However, none of these agents were more effective when compared with cisplatin.

Thus, a need exists for an effective treatment which is relatively non-toxic. This invention satisfies this need and provides related advantages as well.

### SUMMARY

This invention provides a method for inhibiting the growth of a tumor in a cancer patient or treating a cancer patient, wherein the cancer patient has renal insufficiency, the method comprising, or alternatively consisting essentially of, or yet further consisting of, administering to the patient an effective amount of Lipoplatin, thereby inhibiting the growth of the tumor or treating the cancer. In one aspect, the method further comprises, or alternatively consists essentially of, or yet further consists of, administration of an effective amount of a second chemotherapeutic drug. The second chemotherapy can be administered prior to or after the Lipoplatin therapy or simultaneously.

A kit also is provided by Applicant, that provides Lipoplatin alone or in combination with second or other chemotherapeutic drug or anticancer agent, and optionally, instructions for performing the methods of this disclosure.

### DETAILED DESCRIPTION

Throughout this application, the text refers to various embodiments of the present compositions and methods. The various embodiments described are meant to provide a variety of illustrative examples and should not be construed as descriptions of alternative species. Rather it should be noted that the descriptions of various embodiments provided herein may be of overlapping scope. The embodiments discussed herein are merely illustrative and are not meant to limit the scope of the present invention.

Also throughout this disclosure, various publications, patents and published patent specifications are referenced by an identifying. The disclosures of these publications, patents and published patent specifications are hereby incorporated by reference into the present disclosure in their entirety to more fully describe the state of the art to which this invention pertains.

### DEFINITIONS

As used in the specification and claims, the singular form “a,” “an” and “the” include plural references unless the context clearly dictates otherwise. For example, the term “a cell” includes a plurality of cells, including mixtures thereof.

As used herein, the term “comprising” is intended to mean that the compositions and methods include the recited elements, but not excluding others. “Consisting essentially of” when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients. Embodiments defined by each of these transition terms are within the scope of this invention.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above.

All numerical designations, e.g., pH, temperature, time, concentration, and molecular weight, including ranges, are approximations which are varied (+) or (−) by increments of 0.1 or 1.0 as is appropriate. It is to be understood, although not always explicitly stated that all numerical designations are preceded by the term “about” which includes a standard deviation of about 15%, or alternatively about 10% or alternatively about 5%. It also is to be understood, although not always explicitly stated, that the reagents described herein are merely exemplary and that equivalents of such are known in the art.

An “effective amount” is an amount sufficient to effect beneficial or desired results. An effective amount can be administered in one or more administrations, applications or dosages. Such delivery is dependent on a number of variables including the time period for which the individual dosage unit is to be used, the bioavailability of the therapeutic agent, the route of administration, etc. It is understood, however, that specific dose levels of the therapeutic agents of the present invention for any particular subject depends upon a variety of factors including the activity of the specific compound employed, bioavailability of the compound, the route of administration, the age of the animal and its body weight, general health, sex, the diet of the animal, the time of administration, the rate of excretion, the drug combination, and the severity of the particular disorder being treated and form of administration. Treatment dosages generally may be titrated to optimize safety and efficacy. Typically, dosage-effect relationships from in vitro and/or in vivo tests initially can provide useful guidance on the proper doses for patient administration. Studies in animal models generally may be used for guidance regarding effective dosages for treatment of diseases. In general, one will desire to administer an amount of the compound that is effective to achieve a serum level commensurate with the concentrations found to be effective in vitro. Thus, where a compound is found to demonstrate in vitro activity, for example as noted in the Tables discussed below one can extrapolate to an effective dosage for administration in vivo. These considerations, as well as effective formulations and administration procedures are well known in the art and are described in standard textbooks. Consistent with this definition and as used herein, the term “therapeutically effective amount” is an amount sufficient to treat a specified disorder or disease or alternatively to obtain a pharmacological response treating a glioblastoma.

As used herein, “treating” or “treatment” of a disease in a patient refers to (1) preventing the symptoms or disease from occurring in an animal that is predisposed or does not yet display symptoms of the disease; (2) inhibiting the disease or arresting its development; or (3) ameliorating or causing regression of the disease or the symptoms of the disease.

As understood in the art, “treatment” is an approach for obtaining beneficial or desired results, including clinical results. For the purposes of this invention, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. Preferred are compounds that are potent and can be administered locally at very low doses, thus minimizing systemic adverse effects.

As used herein, “surgery” or “surgical resection” refers to surgical removal of a tumor of concern.

“Tumor Recurrence” as used herein and as defined by the National Cancer Institute is cancer that has recurred (come back), usually after a period of time during which the cancer could not be detected. The cancer may come back to the same place as the original (primary) tumor or to another place in the body. It is also called recurrent cancer. “Time to Tumor Recurrence” (TTR) is defined as the time from the date of diagnosis of the cancer to the date of first recurrence, death, or until last contact if the patient was free of any tumor recurrence at the time of last contact. If a patient had not recurred, then TTR was censored at the time of death or at the last follow-up.

“Disease free survival” indicates the length of time after treatment of a cancer or tumor, such as surgery, during which a patient survives with no signs of the cancer or tumor.

“Overall Survival” (OS) intends a prolongation in life expectancy as compared to naïve or untreated individuals or patients.

“Progressive Disease” (PD) intents a disease that is progressing or worsening. For example, with lung cancer, progressive disease can be a 20% growth in the size of the tumor or spread of the tumor since the beginning of treatment.

“Relative Risk” (RR), in statistics and mathematical epidemiology, refers to the risk of an event (or of developing a disease) relative to exposure. Relative risk is a ratio of the probability of the event occurring in the exposed group versus a non-exposed group.

“Monotherapy” as used herein refers to a therapy which is administered by itself.

The term “determine” or “determining” is to associate or affiliate a patient closely to a group or population of patients who likely experience the same or a similar clinical response.

As used herein, the terms “Stage I cancer,” “Stage II cancer,” “Stage III cancer,” and “Stage IV” refer to the TNM staging classification for cancer. Stage I cancer typically identifies that the primary tumor is limited to the organ of origin. Stage II intends that the primary tumor has spread into surrounding tissue and lymph nodes immediately draining the area of the tumor. Stage III intends that the primary tumor is large, with fixation to deeper structures. Stage IV intends that the primary tumor is large, with fixation to deeper structures. See pages 20 and 21, CANCER BIOLOGY, 2^{nd} Ed., Oxford University Press (1987).

“Triple negative breast cancer” intends tumor that was tested for the expression of the markers: estrogen receptor (ER), the progesterone receptor (PR) and herceptin (HER2/neu), and is negative for all three markers.

Lipoplatin™ is a therapeutic composition and its method of making are described in U.S. Pat. Nos. 7,393,478 and 6,511,676, each incorporated by reference herein. The composition is described as a cisplatin micelle containing cisplatin in its aqua form, and obtainable by a method comprising, or alternatively consisting essentially of, or yet further consisting of: a) combining a suitable buffer solution, cisplatin with an effective amount of at least a 30% ethanol solution to form a cisplatin/ethanol solution; and b) combining the solution with a negatively charged phosphatidyl glycerol lipid derivative wherein the molar ratio between cisplatin and the lipid derivative is 1:1 to 1:2, thereby producing a cisplatin mixture in its aqua form in micelles. In one aspect, the ciplatin micelles are obtainable by a method that comprises, or alternatively consists essentially of, or yet further consists of: a) combining a suitable buffer solution, cisplatin with an effective amount of at least 30% ethanol solution to form a cisplatin/ethanol solution; and b) combining the cisplatin/ethanol solution with a negatively charged phosphatidyl glycerol lipid derivative wherein the molar ratio between cisplatin and the lipid derivative is 1:1 to 1:2, thereby producing a cisplatin mixture in its aqua form in micelles. In one aspect, the phosphatidyl glycerol lipid derivative is selected from the group consisting of dipalmitoyl phosphatidyl glycerol (DPPG), dimyristoyl phosphatidyl glycerol (DMPG), dicaproyl phosphatidyl glycerol (DCPG), distearoyl phosphatidyl glycerol (DSPG) and dioleyl phosphatidyl glycerol (DOPG). In another aspect, the molar ratio is 1:1. In a yet further aspect, the method to produce Lipoplatin further comprises, or alternatively consists essentially of, or yet further consists of combining an effective amount of a free fusogenic peptide, a fusogenic peptide-lipid conjugate or a fusogenic peptide-PEG-HSPC conjugate to the mixture of step a) where the fusogenic peptide is derivatized with a stretch of 1-6 negatively-charged amino acids at the N or C-terminus and thus, able to bind electrostatically to the cisplatin mixture in its aqua form. In one aspect, the free fusogenic peptide or fusogenic peptide lipid conjugate comprises, or alternatively consists essentially of, or yet further consists of, DOPE or DOPE/cationic lipid.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents.

The compositions also can include stabilizers and preservatives. For examples of carriers, stabilizers and adjuvants, see Martin (1975) Remington's Pharm. Sci., 15th Ed. (Mack Publ. Co., Easton).

A “subject,” “individual” or “patient” is used interchangeably herein, and refers to a vertebrate, preferably a mammal, more preferably a human. Mammals include, but are not limited to, murines, rats, rabbit, simians, bovines, ovine, porcine, canines, feline, farm animals, sport animals, pets, equine, and primate, particularly human. Besides being useful for human treatment, the present invention is also useful for veterinary treatment of companion mammals, exotic animals and domesticated animals, including mammals, rodents, and the like.

The term administration shall include without limitation, administration by ocular, oral, intra-arterial, parenteral (e.g., intramuscular, intraperitoneal, inhalation, transdermal intravenous, ICV, intracisternal injection or infusion, subcutaneous injection, or implant), by inhalation spray nasal, vaginal, rectal, sublingual, urethral (e.g., urethral suppository) or topical routes of administration (e.g., gel, ointment, cream, aerosol, ocular etc.) and can be formulated, alone or together, in suitable dosage unit formulations containing conventional non-toxic pharmaceutically acceptable carriers, adjuvants, excipients, and vehicles appropriate for each route of administration. The invention is not limited by the route of administration, the formulation or dosing schedule.

A “pathological cell” is one that is pertaining to or arising from disease. Pathological cells can be hyperproliferative. A “hyperproliferative cell” means cells or tissue are dividing and growing at a rate greater than that when the cell or tissue is in a normal or healthy state. Examples of such include, but are not limited to cancer cells.

Hyperproliferative cells also include de-differentiated, immortalized, neoplastic, malignant, metastatic, and cancer cells such as sarcoma cells, leukemia cells, carcinoma cells, or adenocarcinoma cells. Specified cancers include, but are not limited to lung cancer cells, glioblastoma cells, and esophageal carcinoma cells.

A “control” is an alternative subject or sample used in an experiment for comparison purpose. A control can be “positive” or “negative”. For example, where the purpose of the experiment is to determine a correlation of the efficacy of a composition of the invention for the treatment for a particular type of disease or cancer, it is generally preferable to use a positive control (a compound or composition known to exhibit the desired therapeutic effect) and a negative control (a subject or a sample that does not receive the therapy or receives a placebo).

The terms “cancer,” “neoplasm,” and “tumor,” used interchangeably and in either the singular or plural form, refer to cells that have undergone a malignant transformation that makes them pathological to the host organism. Primary cancer cells (that is, cells obtained from near the site of malignant transformation) can be readily distinguished from non-cancerous cells by well-established techniques, particularly histological examination.

The definition of a cancer cell, as used herein, includes not only a primary cancer cell, but also any cell derived from a cancer cell ancestor. This includes metastasized cancer cells, and in vitro cultures and cell lines derived from cancer cells. When referring to a type of cancer that normally manifests as a solid tumor, a “clinically detectable” tumor is one that is detectable on the basis of tumor mass; e.g., by such procedures as CAT scan, magnetic resonance imaging (MRI), X-ray, ultrasound or palpation. Biochemical or immunologic findings alone may be insufficient to meet this definition.

A neoplasm is an abnormal mass or colony of cells produced by a relatively autonomous new growth of tissue. Most neoplasms arise from the clonal expansion of a single cell that has undergone neoplastic transformation. The transformation of a normal to a neoplastic cell can be caused by a chemical, physical, or biological agent (or event) that directly and irreversibly alters the cell genome. Neoplastic cells are characterized by the loss of some specialized functions and the acquisition of new biological properties, foremost, the property of relatively autonomous (uncontrolled) growth. Neoplastic cells pass on their heritable biological characteristics to progeny cells.

The past, present, and future predicted biological behavior, or clinical course, of a neoplasm is further classified as benign or malignant, a distinction of great importance in diagnosis, treatment, and prognosis. A malignant neoplasm manifests a greater degree of autonomy, is capable of invasion and metastatic spread, may be resistant to treatment, and may cause death. A benign neoplasm has a lesser degree of autonomy, is usually not invasive, does not metastasize, and generally produces no great harm if treated adequately.

Cancer is a generic term for malignant neoplasms. Anaplasia is a characteristic property of cancer cells and denotes a lack of normal structural and functional characteristics (undifferentiation).

A tumor is literally a swelling of any type, such as an inflammatory or other swelling, but modem usage generally denotes a neoplasm. The suffix “-oma” means tumor and usually denotes a benign neoplasm, as in fibroma, lipoma, and so forth, but sometimes implies a malignant neoplasm, as with so-called melanoma, hepatoma, and seminoma, or even a non-neoplastic lesion, such as a hematoma, granuloma, or hamartoma. The suffix “-blastoma” denotes a neoplasm of embryonic cells, such as neuroblastoma of the adrenal or retinoblastoma of the eye.

Histogenesis is the origin of a tissue and is a method of classifying neoplasms on the basis of the tissue cell of origin. Adenomas are benign neoplasms of glandular epithelium. Carcinomas are malignant tumors of epithelium. Sarcomas are malignant tumors of mesenchymal tissues. One system to classify neoplasia utilizes biological (clinical) behavior, whether benign or malignant, and the histogenesis, the tissue or cell of origin of the neoplasm as determined by histologic and cytologic examination. Neoplasms may originate in almost any tissue containing cells capable of mitotic division. The histogenetic classification of neoplasms is based upon the tissue (or cell) of origin as determined by histologic and cytologic examination.

“Inhibiting” tumor growth indicates a growth state that is curtailed compared to growth without any therapy. Tumor cell growth can be assessed by any means known in the art, including, but not limited to, measuring tumor size, determining whether tumor cells are proliferating using a ³H-thymidine incorporation assay, or counting tumor cells.

“Suppressing” tumor cell growth means any or all of the following states: slowing, delaying, and “suppressing” tumor growth indicates a growth state that is curtailed when stopping tumor growth, as well as tumor shrinkage.

The term “culturing” refers to the in vitro propagation of cells or organisms on or in media of various kinds. It is understood that the descendants of a cell grown in culture may not be completely identical (morphologically, genetically, or phenotypically) to the parent cell. By “expanded” is meant any proliferation or division of cells.

As used herein, the term “renal insufficiency” (also called in some aspect, renal failure) intends when insufficient kidney function exists to maintain a normal state of health.

### DESCRIPTIVE EMBODIMENTS

This invention provides a method for inhibiting the growth of a tumor in a cancer patient or treating a cancer patient, wherein the cancer patient has renal insufficiency, the method comprising, or alternatively consisting essentially of, or yet further consisting of, administering to the patient an effective amount of Lipoplatin, thereby inhibiting the growth of the tumor or treating the cancer. In one aspect, the method further comprises, or alternatively consists essentially of, or yet further consists of, administration of an effective amount of a second chemotherapeutic. The second chemotherapy can be administered prior to or after the Lipoplatin therapy or simultaneously. As used herein, the term “chemotherapeutic” intends small molecule and large molecule (biologic-based, e.g., antibody based) therapeutics.

The effective amount is administered in a dose determined by the treating physician to provide the most therapeutic benefit to the patient and will vary with the patient, the cancer and the prior treatments and duration of the therapy.

The methods are useful to inhibit the growth of a solid tumor or treat a cancer from the group of metastatic or non-metastatic lung cancer, gastrointestinal cancer, bladder cancer, non-small cell lung cancer (NSCLC), breast cancer, Triple-negative breast cancer, gastric cancer, head and neck cancer, colon cancer, colorectal cancer, rectal cancer, mesothelioma, pancreatic cancer, brain cancer, (glioblastoma multiform or metastases) or ovarian cancer.

In a further aspect, the method further comprises, or alternatively consists essentially of, or yet further consists of, administering an effective amount of a second chemotherapeutic agent. Non-limiting examples of are described herein, e.g., one or more of oxaliplatin, paclitaxel, taxol, taxane, 5-Fluoropyrimidine (5-FU), vinorelbine or gemcitabine and equivalents of each thereof.

The method can be used as a first line, a second line or a third line therapy for the patient. In one aspect, the patient previously underwent surgical resection and/or radiotherapy. In a further aspect, the patient was previously treated with first line oxaliplatin therapy. In one aspect, Lipoplatin is administered with paclitaxel or an equivalent thereof. In another aspect, Lipoplatin is administered with 5-FU or an equivalent thereof. In another aspect, Lipoplatin is administered with gemcitabine or an equivalent thereof. In one aspect the treatment is administered as a first or second line therapy. In another aspect, the treatment is administered as a second or third line therapy.

Any suitable route of administration is acceptable, and can be determined by the treating physician. Non-limiting examples include intravenously or by inhalation therapy. In one aspect of the invention, the second chemotherapeutic drug is a DNA alkylating agent which attaches an alkyl group to DNA. Such agents are well known in the art and are used to treat a variety of tumors. Non-limiting examples of a DNA alkylating agents are Nitrogen mustards, such as Mechlorethamine, Cyclophosphamide (Ifosfamide, Trofosfamide), Chlorambucil (Melphalan, Prednimustine), Bendamustine, Uramustine and Estramustine; Nitrosoureas, such as Carmustine (BCNU), Lomustine (Semustine), Fotemustine, Nimustine, Ranimustine and Streptozocin; Alkyl sulfonates, such as Busulfan (Mannosulfan, Treosulfan); Aziridines, such as Carboquone, ThioTEPA, Triaziquone, Triethylenemelamine; Hydrazines (Procarbazine); Triazenes such as Dacarbazine and Temozolomide; Altretamine and Mitobronitol.

In another aspect of the invention, the second chemotherapeutic drug is a platinum based compound which is a subclass of DNA alkylating agents. Such agents are well known in the art and are used to treat a variety of cancers, such as, lung cancers, head and neck cancers, ovarian cancers, colorectal cancer and prostate cancer. Non-limiting examples of such agents include Carboplatin, Cisplatin, Nedaplatin, Oxaliplatin, Triplatin tetranitrate, Satraplatin, Aroplatin, Lobaplatin, and JM-216. (see McKeage et al. (1997) J. Clin. Oncol. 201:1232-1237 and in general, CHEMOTHERAPY FOR GYNECOLOGICAL NEOPLASM, CURRENT THERAPY AND NOVEL APPROACHES, in the Series Basic and Clinical Oncology, Angioli et al. Eds., 2004).

“Oxaliplatin” (Eloxatin®) is a platinum-based chemotherapy drug in the same family as cisplatin and carboplatin. It is typically administered in combination with fluorouracil and leucovorin in a combination known as FOLFOX for the treatment of colorectal cancer. Compared to cisplatin the two amine groups are replaced by cyclohexyldiamine for improved antitumour activity. The chlorine ligands are replaced by the oxalato bidentate derived from oxalic acid in order to improve water solubility. Equivalents to Oxaliplatin are known in the art and include without limitation cisplatin, carboplatin, aroplatin, lobaplatin, nedaplatin, and JM-216 (see McKeage et al. (1997) J. Clin. Oncol. 201:1232-1237 and in general, CHEMOTHERAPY FOR GYNECOLOGICAL NEOPLASM, CURRENT THERAPY AND NOVEL APPROACHES, in the Series Basic and Clinical Oncology, Angioli et al. Eds., 2004).

In one aspect of the invention, the second chemotherapeutic drug is a topoisomerase inhibitor which is an agent that interferes with the action of topoisomerase enzymes (topoisomerase I and II). Topoisomerases are enzymes that control the changes in DNA structure by catalyzing the breaking and rejoining of the phosphodiester backbone of DNA. Such agents are well known in the art. Non-limiting examples of Topoisomerase I inhibitors include Campothecine derivatives including CPT-11/Irinotecan, SN-38, APC, NPC, camptothecin, topotecan, exatecan mesylate, 9-nitrocamptothecin, 9-aminocamptothecin, lurtotecan, rubitecan, silatecan, gimatecan, diflomotecan, extatecan, BN-80927, DX-8951f, and MAG-CPT as described in Pommier (2006) Nat. Rev. Cancer 6(10):789-802 and U.S. Patent Appl. No. 2005/0250854; Protoberberine alkaloids and derivatives thereof including berberrubine and coralyne as described in Li et al. (2000) Biochemistry 39(24):7107-7116 and Gatto et al. (1996) Cancer Res. 15(12):2795-2800; Phenanthroline derivatives including Benzo[i]phenanthridine, Nitidine, and fagaronine as described in Makhey et al. (2003) Bioorg. Med. Chem. 11(8):1809-1820; Terbenzimidazole and derivatives thereof as described in Xu (1998) Biochemistry 37(10):3558-3566; and Anthracycline derivatives including Doxorubicin, Daunorubicin, and Mitoxantrone as described in Foglesong et al. (1992) Cancer Chemother. Pharmacol. 30(2):123-125, Crow et al. (1994) J. Med. Chem. 37(19):3191-3194, and (Crespi et al. (1986) Biochem. Biophys. Res. Commun. 136(2):521-8.

In one aspect of the invention, the topoisomerase I inhibitors can be selected from the group of, but not limited to, Campothecine derivatives including CPT-11/Irinotecan, SN-38, APC, NPC, camptothecin, topotecan, exatecan mesylate, 9-nitrocamptothecin, 9-aminocamptothecin, lurtotecan, rubitecan, silatecan, gimatecan, diflomotecan, extatecan, BN-80927, DX-8951f, and MAG-CPT as described in Pommier (2006) Nat. Rev. Cancer 6(10):789-802 and US Patent Appl. No. 2005/0250854; Protoberberine alkaloids and derivatives thereof including berberrubine and coralyne as described in Li et al. (2000) Biochemistry 39(24):7107-7116 and Gatto et al. (1996) Cancer Res. 15(12):2795-2800; Phenanthroline derivatives including Benzo[i]phenanthridine, Nitidine, and fagaronine as described in Makhey et al. (2003) Bioorg. Med. Chem. 11(8):1809-1820; Terbenzimidazole and derivatives thereof as described in Xu (1998) Biochemistry 37(10):3558-3566; and Anthracycline derivatives including Doxorubicin, Daunorubicin, and Mitoxantrone as described in Foglesong et al. (1992) Cancer Chemother. Pharmacol. 30(2):123-125, Crow et al. (1994) J. Med. Chem. 37(19):3191-3194, and (Crespi et al. (1986) Biochem. Biophys. Res. Commun. 136(2):521-8, will be used in combination therapy with antibody based chemotherapy described above to treat patients identified with the appropriate genetic markers.

Irinotecan (CPT-11) is sold under the tradename of Camptosar®. It is a semi-synthetic analogue of the alkaloid camptothecin, which is activated by hydrolysis to SN-38 and targets topoisomerase I. Chemical equivalents are those that inhibit the interaction of topoisomerase I and DNA to form a catalytically active topoisomerase I-DNA complex. Chemical equivalents inhibit cell cycle progression at G2-M phase resulting in the disruption of cell proliferation.

In another aspect, some second chemotherapeutic drugs inhibit Topoisomerase II and have DNA intercalation activity such as, but not limited to, Anthracyclines (Aclarubicin, Daunorubicin, Doxorubicin, Epirubicin, Idarubicin, Amrubicin, Pirarubicin, Valrubicin, Zorubicin) and Antracenediones (Mitoxantrone and Pixantrone).

In one aspect of the invention, Topoisomerase II inhibitors include, but are not limited to Etoposide and Teniposide.

In another aspect of the invention, the second chemotherapeutic drug is a dual topoisomerase I and II inhibitors selected from the group of, but not limited to, Saintopin and other Naphthecenediones, DACA and other Acridine-4-Carboxamindes, Intoplicine and other Benzopyridoindoles, TAS-103 and other 7H-indeno[2,1-c]Quinoline-7-ones, Pyrazoloacridine, XR 11576 and other Benzophenazines, XR 5944 and other Dimeric compounds, and Anthracenyl-amino Acid Conjugates as described in Denny and Baguley (2003) Curr. Top. Med. Chem. 3(3):339-353. In one aspect, they can be used in combination therapy with antibody based chemotherapy described above to treat patients identified with the appropriate genetic markers.

“Lapatinib” (Tykerb®) is an oncolytic dual EGFR and erbB-2 inhibitor. Lapatinib has been investigated as an anticancer monotherapy, as well as in combination with trastuzumab, capecitabine, letrozole, paclitaxel and FOLFIRI (irinotecan, 5-fluorouracil and leucovorin), in a number of clinical trials. It is currently in phase III testing for the oral treatment of metastatic breast, head and neck, lung, gastric, renal and bladder cancer. A chemical equivalent of lapatinib is a small molecule or compound that is a tyrosine kinase inhibitor or alternatively a HER-1 inhibitor or a HER-2 inhibitor. Several TKIs have been found to have effective antitumor activity and have been approved or are in clinical trials. Examples of such include, but are not limited to Zactima (ZD6474), Iressa (gefitinib) and Tarceva (erlotinib), imatinib mesylate (STI571; Gleevec), erlotinib (OSI-1774; Tarceva), canertinib (CI 1033), semaxinib (SU5416), vatalanib (PTK787/ZK222584), sorafenib (BAY 43-9006), sutent (SU11248) and leflunomide (SU101).

A biological equivalent of lapatinib is a peptide, antibody or antibody derivative thereof that is a HER-1 inhibitor and/or a HER-2 inhibitor. Examples of such include but are not limited to the humanized antibody trastuzumab and Herceptin.

In another aspect of the invention, the second chemotherapeutic drug is an antimetabolite agent which inhibits the use of a metabolite, i.e. another chemical that is part of normal metabolism. In cancer treatment, antimetabolites interfere with DNA production, thus cell division and growth of the tumor. Non-limiting examples of these agents are Folic acid based, i.e. dihydrofolate reductase inhibitors, such as Aminopterin, Methotrexate and Pemetrexed; thymidylate synthase inhibitors, such as Raltitrexed, Pemetrexed; Purine based, i.e. an adenosine deaminase inhibitor, such as Pentostatin, a thiopurine, such as Thioguanine and Mercaptopurine, a halogenated/ribonucleotide reductase inhibitor, such as Cladribine, Clofarabine, Fludarabine, or a guanine/guanosine: thiopurine, such as Thioguanine; or Pyrimidine based, i.e. cytosine/cytidine: hypomethylating agent, such as Azacitidine and Decitabine, a DNA polymerase inhibitor, such as Cytarabine, a ribonucleotide reductase inhibitor, such as Gemcitabine, or a thymine/thymidine: thymidylate synthase inhibitor, such as a Fluorouracil (5-FU).

Fluorouracil (5-FU) belongs to the family of therapy drugs call pyrimidine based antimetabolites. 5-FU is transformed into different cytotoxic metabolites that are then incorporated into DNA and RNA thereby inducing cell cycle arrest and apoptosis. It is a pyrimidine analog, which is transformed into different cytotoxic metabolites that are then incorporated into DNA and RNA thereby inducing cell cycle arrest and apoptosis. Chemical equivalents are pyrimidine analogs which result in disruption of DNA replication. Chemical equivalents inhibit cell cycle progression at S phase resulting in the disruption of cell cycle and consequently apoptosis. Equivalents to 5-FU include prodrugs, analogs and derivative thereof such as 5′-deoxy-5-fluorouridine (doxifluroidine), 1-tetrahydrofuranyl-5-fluorouracil (ftorafur), Capecitabine (Xeloda), S-1 (MBMS-247616, consisting of tegafur and two modulators, a 5-chloro-2,4-dihydroxypyridine and potassium oxonate), ralititrexed (tomudex), nolatrexed (Thymitaq, AG337), LY231514 and ZD9331, as described for example in Papamicheal (1999) The Oncologist 4:478-487.

Capecitabine and Tegafur are examples of chemical equivalents of 5-FU. It is a prodrug of (5-FU) that is converted to its active form by the tumor-specific enzyme PynPase following a pathway of three enzymatic steps and two intermediary metabolites, 5′-deoxy-5-fluorocytidine (5′-DFCR) and 5′-deoxy-5-fluorouridine (5′-DFUR). Capecitabine is marketed by Roche under the trade name Xeloda®.

Leucovorin (Folinic acid) is an adjuvant used in cancer therapy. It is used in synergistic combination with 5-FU to improve efficacy of the chemotherapeutic agent. Without being bound by theory, addition of Leucovorin is believed to enhance efficacy of 5-FU by inhibiting thymidylate synthase. It has been used as an antidote to protect normal cells from high doses of the anticancer drug methotrexate and to increase the antitumor effects of fluorouracil (5-FU) and tegafur-uracil. It is also known as citrovorum factor and Wellcovorin. This compound has the chemical designation of L-Glutamic acid N[4[[(2-amino-5-formyl1,4,5,6,7,8hexahydro4oxo6-pteridinyl)methyl]amino]benzoyl], calcium salt (1:1).

Examples of vincalkaloids, include, but are not limited to vinblastine, Vincristine, Vinflunine, Vindesine and Vinorelbine.

Examples of taxanes include, but are not limited to docetaxel, Larotaxel, Ortataxel, Paclitaxel and Tesetaxel. An example of an epothilone is iabepilone.

Examples of enzyme inhibitors include, but are not limited to farnesyltransferase inhibitors (Tipifarnib); CDK inhibitor (Alvocidib, Seliciclib); Proteasome inhibitor (Bortezomib); Phosphodiesterase inhibitor (Anagrelide); IMP dehydrogenase inhibitor (Tiazofurine); and Lipoxygenase inhibitor (Masoprocol).

Examples of tyrosine kinase inhibitors include, but are not limited to ErbB: HER1/EGFR (Erlotinib, Gefitinib, Lapatinib, Vandetanib, Sunitinib, Neratinib); HER2/neu (Lapatinib, Neratinib); RTK class III: C-kit (Axitinib, Sunitinib, Sorafenib); FLT3 (Lestaurtinib); PDGFR (Axitinib, Sunitinib, Sorafenib); and VEGFR (Vandetanib, Semaxanib, Cediranib, Axitinib, Sorafenib); bcr-abl (Imatinib, Nilotinib, Dasatinib); Src (Bosutinib) and Janus kinase 2 (Lestaurtinib).

PTK/ZK is a “small” molecule tyrosine kinase inhibitor with broad specificity that targets all VEGF receptors (VEGFR), the platelet-derived growth factor (PDGF) receptor, c-KIT and c-Fms. Drevs (2003) Idrugs 6(8):787-794. PTK/ZK is a targeted drug that blocks angiogenesis and lymphangiogenesis by inhibiting the activity of all known receptors that bind VEGF including VEGFR-1 (Flt-1), VEGFR-2 (KDR/Flk-1) and VEGFR-3 (Flt-4). The chemical names of PTK/ZK are 1-[4-Chloroanilino]-4-[4-pyridylmethyl]phthalazine Succinate or 1-Phthalazinamine, N-(4-chlorophenyl)-4-(4-pyridinylmethyl)-, butanedioate (1:1). Synonyms and analogs of PTK/ZK are known as Vatalanib, CGP79787D, PTK787/ZK 222584, CGP-79787, DE-00268, PTK-787, PTK-787A, VEGFR-TK inhibitor, ZK 222584 and ZK.

Additional examples of second chemotherapeutic agents and combination therapies include, but are not limited to amsacrine, Trabectedin, retinoids (Alitretinoin, Tretinoin), Arsenic trioxide, asparagine depleter (Asparaginase/Pegaspargase), Celecoxib, Demecolcine, Elesclomol, Elsamitrucin, Etoglucid, Lonidamine, Lucanthone, Mitoguazone, Mitotane, Oblimersen, Temsirolimus, and Vorinostat.

“FOLFOX” is an abbreviation for a type of combination therapy that is used to treat colorectal cancer. It includes 5-FU, oxaliplatin and leucovorin. Information regarding this treatment is available on the National Cancer Institute's web site, cancer.gov, last accessed on Jan. 16, 2008.

“FOLFOX/BV” is an abbreviation for a type of combination therapy that is used to treat colorectal cancer. This therapy includes 5-FU, oxaliplatin, leucovorin and Bevacizumab. Furthermore, “XELOX/BV” is another combination therapy used to treat colorectal cancer, which includes the prodrug to 5-FU, known as Capecitabine (Xeloda) in combination with oxaliplatin and bevacizumab. Information regarding these treatments are available on the National Cancer Institute's web site, cancer.gov or from the National Comprehensive Cancer Network's web site, nccn.org, last accessed on May 27, 2008. Examples of second chemotherapeutics or anticancer drugs include therapeutic antibodies include, but are not limited to anti-HER1/EGFR (Cetuximab, Panitumumab); Anti-HER2/neu (erbB2) receptor (Trastuzumab); Anti-EpCAM (Catumaxomab, Edrecolomab) Anti-VEGF-A (Bevacizumab); Anti-CD20 (Rituximab, Tositumomab, Ibritumomab); Anti-CD52 (Alemtuzumab); and Anti-CD33 (Gemtuzumab), as well as biological equivalents thereof.

Bevacizumab is sold under the trade name Avastin by Genentech. It is a humanized monoclonal antibody that binds to and inhibits the biologic activity of human vascular endothelial growth factor (VEGF). Biological equivalent antibodies are identified herein as modified antibodies and those which bind to the same epitope of the antigen, prevent the interaction of VEGF to its receptors (Flt01, KDR a.k.a. VEGFR2) and produce a substantially equivalent response, e.g., the blocking of endothelial cell proliferation and angiogenesis.

In one aspect, the “chemical equivalent” means the ability of the chemical to selectively interact with its target protein, DNA, RNA or fragment thereof as measured by the inactivation of the target protein, incorporation of the chemical into the DNA or RNA or other suitable methods. Chemical equivalents include, but are not limited to, those agents with the same or similar biological activity and include, without limitation a pharmaceutically acceptable salt or mixtures thereof that interact with and/or inactivate the same target protein, DNA, or RNA as the reference chemical.

In one aspect, the “biological equivalent” means the ability of the antibody to selectively bind its epitope protein or fragment thereof as measured by ELISA or other suitable methods. Biologically equivalent antibodies include, but are not limited to, those antibodies, peptides, antibody fragments, antibody variant, antibody derivative and antibody mimetics that bind to the same epitope as the reference antibody. An example of an equivalent Bevacizumab antibody is one which binds to and inhibits the biologic activity of human vascular endothelial growth factor (VEGF).

A kit also is provided by Applicant, that provides Lipoplatin alone or in combination with second or other chemotherapeutic drug or anticancer agent (as described above), and optionally, instructions for performing the methods of this disclosure.

### EXPERIMENTAL

### Materials and Methods

### Lipoplatin™

Lipoplatin™ is a therapeutic composition and its method of making are described in U.S. Pat. No. 7,393,478, incorporated by reference herein. Briefly, for the sake of completeness, Lipoplatin can be prepared by (step A) mixing cisplatin (in powder or other form) with DPPG (dipalmitoyl phosphatidyl glycerol) or other negatively-charged lipid molecules at a 1:1 to 1:2 molar ratio in at least a 30% ethanol, 0.1 M Tris HCl, pH 7.5 solution. Variations in the molar ratio between cisplatin and DPPG are also of therapeutic value targeting different tissues. In step (B), the composition is heated to 50° C. During steps A and B. the initial powder suspension, which tends to give a precipitate of the yellow cisplatin powder, is converted into a gel (colloidal) form; during steps A and B there is conversion of cisplatin to its aqua form (by hydrolysis of the chloride atoms and their replacement by water molecules bound to the platin) which is positively-charged and is the active form of cisplatin endowed with the antineoplastic activity; the aqua cisplatin is simultaneously complexed with the negatively-charged lipid into micelles in 30% ethanol. This cisplatin-DPPG electrostatic complex has already improved properties over free cisplatin in tumor eradication. (Step C). The properties of the complex (and of the final formulation after step D, see below) in passing through the tumor cell membrane after reaching its target are improved by addition of peptides and other molecules that give to the complex this property. (Step D) The cisplatin-DPPG micelle complex is converted into liposomes encapsulating the cisplatin-DPPG-monolayer (see FIG. 1 top of U.S. Pat. No. 7,393,478) or to other type of complexes by direct addition of premade liposomes followed by dialysis against saline and extrusion through membranes to downsize these to 100-160 nm in diameter (FIG. 1 bottom of U.S. Pat. No. 7,393,478). It is the lipid composition of added liposomes that determines the composition of the outer surface of our final cisplatin formulation. Variations in step (A) permit encapsulation of doxorubicin and other positively charged antineoplastic compounds. Addition of positively charged groups to neutral or negatively-charged compounds allows their encapsulation similarly into liposomes.

16 patients with lung cancer, 10 patients with gastrointestinal cancer and 16 patients with bladder cancer were recruited in this study. All 16 bladder cancer patients had renal insufficiency (creatinine levels 1.6 to 4.0 mg/dl). Lung cancer patients received Lipoplatin plus paclitaxel (as 2^{nd} or 3^{rd} line treatment), patients with gastrointestinal cancer received Lipoplatin and 5-FU (as 2^{nd} or 3^{rd} line treatment), while all 16 bladder cancer patients received Lipoplatin and gemcitabine as 1^{st} or 2^{nd} line treatment. Chemotherapy regimens containing cisplatin are known to increase serum creatinine because of renal toxicity. In contrast, Lipoplatin did not cause any increase in creatinine levels in any of the patients treated in this study. More importantly, in 10/16 bladder cancer patients with renal insufficiency, the blood urea and serum creatinine levels decreased, towards normal levels; this reduction was observed in these patients who had had a urination obstruction, which after treatment returned to normal.

Absence of creatinine elevation in response to a Lipoplatin-containing regimen occurred in the absence of dialysis for all 42 patients. Thus, the results of this study are consistent with the very low nephrotoxicity profile of Lipoplatin that has been documented in previous clinical trials. Moreover, the results extend the favorable nephrotoxicity profile of Lipoplatin in patients with renal insufficiency, suggesting that Lipoplatin may be the cisplatin of choice in this patient population.

As expected, some toxicity was observed in the study, namely Grade 1 & 2 myelotoxicity; however, it was mild and did not necessitate the use of growth factors. With regards to efficacy, a Complete Response was observed in 5 patients with bladder cancer, Partial Response in 15 patients (8 with bladder, 2 with NSCLC and 5 with GI tract cancers) and Stable Disease was observed in 14 patients (3 with bladder, 6 with NSCLC and 5 with GI tract cancers). Only 8 of 42 patients had progressive disease.

Thus, it should be understood that although the present disclosure has been specifically disclosed by preferred embodiments and optional features, modification, improvement and variation of the disclosure embodied therein herein disclosed may be resorted to by those skilled in the art, and that such modifications, improvements and variations are considered to be within the scope of this disclosure. The materials, methods, and examples provided here are representative of preferred embodiments, are exemplary, and are not intended as limitations on the scope of the disclosure.

The disclosure has been described broadly and generically herein. Each of the narrower species and subgeneric groupings falling within the generic disclosure also form part of the disclosure. This includes the generic description of the disclosure with a proviso or negative limitation removing any subject matter from the genus, regardless of whether or not the excised material is specifically recited herein.

In addition, where features or aspects of the disclosure are described in terms of Markush groups, those skilled in the art will recognize that the disclosure is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

1. A method for inhibiting the growth of a tumor in a cancer patient or treating a cancer patient, wherein the cancer patient has renal insufficiency, the method comprising administering to the patient an effective amount of Lipoplatin, thereby inhibiting the growth of the tumor or treating the cancer.

2. The method of claim 1, further comprising administering an effective amount of a second chemotherapeutic.

3. The method of claim 2, wherein the second chemotherapy is administered prior to or after the Lipoplatin therapy or simultaneously with Lipoplatin therapy.

4. The methods of any one of claims 1 to 3, wherein the tumor or cancer is from the group of metastatic or non-metastatic lung cancer, gastrointestinal cancer, bladder cancer, non-small cell lung cancer (NSCLC), breast cancer, Triple-negative breast cancer, gastric cancer, head and neck cancer, colon cancer, colorectal cancer, rectal cancer, mesothelioma, pancreatic cancer, brain cancer, (glioblastoma multiform or metastases) or ovarian cancer.

5. A kit comprising a Lipoplatin and instructions for the method of any one of claims 1 to 3.

6. The kit of claim 5, further comprising a second chemotherapeutic agent.
